(19) United States
(12) Patent Application Publication
(10) Pub. No.: US 2023/0338265 A1
(43) Pub. Date: Oct. 26, 2023

(54) NOVEL USE OF SACCHARIDE ISOMERATE

(71) Applicant: DSM IP Assets B.V., Heerlen (NL)

(72) Inventors: Joshua CLAYPOOL, Kaiseraugst (CH); Riccardo SFRISO, Kaiseraugst (CH)

(21) Appl. No.: 18/025,380

(22) PCT Filed: Sep. 10, 2021
(86) PCT No.: PCT/EP2021/074914

§ 371 Date: Mar. 8, 2023

Related U.S. Application Data
(60) Provisional application No. 63/076,529, filed on Sep. 10, 2020.

(30) Foreign Application Priority Data
Oct. 16, 2020 (EP) 20202294.3

(51) Int. Cl. A61K 8/73; A61Q 17/00

(52) U.S. Cl. CPC A61K 8/73; A61Q 17/005

(57) ABSTRACT

The invention relates to a new use of saccharide isomerate for enriching the abundance of Paracoccus marcusii within the skin microbiome of an individual.

The invention relates to a new use of saccharide isomerate for enriching the abundance of Paracoccus marcusii in the skin microbiome of an individual.

It is well known that the surface of the skin is colonized by a great variety of microorganisms which form the skin microbiome (often also called skin microbiota). Resident microbiota are found in the upper parts of the epidermis and congregated in and around the hair follicle. The majority of the microorganisms on human skin are commensal or mutualistic bacteria. However, the microbiota may also include or be exposed to pathogenic microorganisms such as Brevibacterium casei (B. casei) or Rothia mucilaginosa (R. mucilaginosa) which are part of a group of opportunistic bacteria defined as “coryneforms” increasingly implicated in significant infections. Commensals generally live in peaceful coexistence with the host while benefiting from the sheltered ecological niche. Mutualistic microbes offer benefits to their host. Pathogenic microbes may be harmful to their host, in particular if their presence exceeds certain thresholds.

Carotenoids such as astaxanthin are known to exert benefits on skin homeostasis, such as e.g. an improvement of wrinkled skin and skin elasticity. However an inherent feature of carotenoids is their intense yellow to red color. Thus, even when used in small amounts, carotenoids tend to intensively stain finished product. Stained skin care products are, however, not well accepted by end consumers. Thus, even though their cosmetic benefits have been proven for many years, carotenoids are not widely used in the cosmetic industry. Thus, there is an ongoing need for alternative means to topically supply carotenoids such as astaxanthin to the skin.

Paracoccus marcusii (P. marcusii) is a gram negative coccus which naturally occurs on human skin. Furthermore, Paracoccus marcusii is known to produce carotenoids, mainly astaxanthin and is thus considered a mutualistic skin microbe.

P. marcusii has furthermore been reported to be a hydrocarbons degrader, especially a degrader of polyaromatic hydrocarbons (PAHs) and a producer of biosurfactants. PAHs are known to be adsorbed onto the skin as a result of environmental pollution and are potentially carcinogenic.

Surprisingly it has now been found that saccharide isomerate increases the differential abundance of P. marcusii in the skin microbiome of body skin as it is positively associated with the presence thereof and can thus be used to foster the supply of carotenoids to the body skin as well as to degrade polyaromatic hydrocarbons after adsorption thereof onto the body skin, e.g. due to environmental pollution.

Thus, in a first embodiment the present invention relates to a method to increase the differential abundance of P. marcusii in the skin microbiome of the body skin of an individual, said method comprising the step of topically administering an effective amount of saccharide isomerate to an external surface comprising body skin of the human body in need thereof.

The method and uses according the present invention are preferably used to foster the supply of carotenoids such as astaxanthin to the external surface of body skin of the human body and/or to degrade polyaromatic hydrocarbons after adsorption thereof onto the external surface of the human body.

It has furthermore been found, that saccharide isomerate at the same time also significantly decreases the differential abundance of the pathogenic microbes B. casei and R. mucilaginosa in the skin microbiome.

Thus, in another embodiment, the present invention relates to a method according to the present invention, wherein at the same time the differential abundance of B. casei and/or R. mucilaginosa, preferably of both in the skin microbiome are decreased.

In a further embodiment, the present invention also provides a method for providing protection to an external surface of the human body by increasing the differential abundance of P. marcusii in the skin microbiome of body skin, said method comprising the step of topically applying saccharide isomerate to said external surface.

In a further embodiment, the present invention relates to a method to supply carotenoids such as astaxanthin to an external surface comprising body skin of the human body, said method encompassing the step of topically administering saccharide isomerate to an external surface comprising body skin in need thereof.

In another embodiment, the present invention relates to a method to degrade polyaromatic hydrocarbons adsorbed onto an external surface of the human body comprising body skin, said method encompassing the step of topically administering saccharide isomerate to an external surface comprising body skin in need thereof.

In another embodiment, the present invention relates to the use of saccharide isomerate respectively a topical composition comprising saccharide isomerate for increasing the differential abundance of P. marcusii in the skin microbiome of body skin of an individual in need thereof.

The term external surface of the human body generally encompasses the skin as well as the scalp (including hair and axilla) and the oral cavity. In all embodiments of the present invention the external surface of the human body treated according to the present invention is the body skin.

The term ‘body skin’ as used herein refers to the skin of the arms, the torso, the lower parts of the body such as the legs and the feet, not encompassing the face and the scalp. Particularly preferred is the skin of the arms such as the (volar) forearms.

The term ‘skin microbiome’ as used herein refers to the group of microbes which colonize a defined skin area of an individual, such as e.g. the forehead, the forearm, the cheek or the scalp, without being limited thereto.

The term ‘differential abundance’ as used herein, refers to the logarithm of the fold change in abundance of a taxa between two conditions. This analysis method, giving differentials as output, allows to identify microbiome taxa associated with certain biological or clinical conditions. Differentials can be ranked and sorted from lowest to highest. These “rankings” give information on the relative associations of features with a given covariate (i.e. treatment or time).

The ‘differential abundance’ as used herein is determined by the use of a software called Songbird as outlined in Morton et al. (Nat. Commun., 2019. 10(1): p. 2719), which is included herein by reference, which allows to build a statistical model testing for differences between i.e. treatments and compute differentials starting from relative abundance data coming from 16S rRNA sequencing. The output is a file containing the ranks of the features for certain metadata categories. The higher the rank, the more associated it is with that category (i.e. treatment).

The term ‘increase respectively decrease in the differential abundance’ as used herein is defined as the increase respectively decrease of the differential abundance of a specific microbe when compared to the differential abundance on the respective symmetric skin area placebo-treated of the same individual. Saccharide isomerate (CAS 100843-69-4) is a well-known cosmetic agent with unique binding mechanism to the skin used for short and long-lasting moisturization. Saccharide isomerate is e.g. commercially available under the trademark PENTAVITIN® (from DSM Nutritional Products Ltd) or under the tradename Waterin (from Clariant), under the tradenames ‘EPS3 Powder’, EPS4 Powder’, EPS5 Powder’, and ‘EPS15 Powder (all from Codif) and under the tradename Hyanify from Lipotec. The most preferred saccharide isomerate in all embodiments of the present invention is a saccharide isomerate consisting essentially of
a) 1.5 to 4 wt.-%, preferably 2 to 3 wt.-% of psicose,
b) 1 to 5 wt.-%, preferably 1.5 to 2.5 wt.-% of mannose
c) 10 to 30 wt.-%, preferably 15 to 25 wt.-% of fructose,
d) 20 to 60 wt.-%, preferably 10 to 30 wt.-% of glucose, and
e) 0 to 5 wt.-%, preferably 0 to 1 wt.-% of galactose.

The term ‘consisting essentially of’ as used herein means that the total amount of the listed ingredients ideally sums up to 100 wt.-%. It is however not excluded that small amount of unknown (sugar) impurities derived from the isomerisation process may be present, however only in amounts of up to 5 wt.-%, preferably up to 2.5 wt.-%.

Said saccharide isomerate is preferably prepared by isomerisation of plant derived glucose and is commercially available as PENTAVITIN® from DSM Nutritional products Ltd.

The saccharide isomerate is preferably administered in the form of a topical composition comprising an effective amount of the saccharide isomerate and a cosmetically acceptable carrier.

The term ‘an effective amount’ refers to an amount necessary to obtain the desired physiological effect. The physiological effect may be achieved by one application dose or by repeated applications. The dosage administered may, of course, vary depending upon known factors, such as the physiological characteristics of the particular composition comprising the saccharide isomerate and its mode and route of administration; the age, health and weight of the recipient; the kind of concurrent treatment; the frequency of treatment; and the effect desired and can be adjusted by a person skilled in the art.

Generally, the amount of the saccharide isomerate in the topical compositions according to the present invention is selected in the range from 0.01 to 10 wt.-%, preferably in the range from 0.1 to 7.5 wt.-%, more preferably in the range from 0.2 to 5 wt.-%, based on the total weight of the topical composition. Further suitable ranges are from 0.25 to 2.5 wt.-% and from 0.5 to 2 wt.-%.

The term ‘cosmetically acceptable carrier’ (also referred to herein as carrier) refers to all vehicles/carriers conventionally used in topical cosmetic compositions, i.e. which are suitable for topical application to the keratinous tissue, have good aesthetic properties, are compatible with the actives present in the composition, and will not cause any unreasonable safety or toxicity concerns. Such carriers are well-known to one of ordinary skill in the art.

The exact amount of carrier will depend upon the actual level of the saccharide isomerate and of any other optional ingredients that one of ordinary skill in the art would classify as distinct from the carrier (e.g., other active ingredients)

In an advantageous embodiment, the topical compositions according to the present invention comprise from about 50% to about 99%, preferably from about 60% to about 98%, more preferably from about 70% to about 98%, such as in particular from about 80% to about 95% of a carrier, based on the total weight of the topical composition.

In a particular advantageous embodiment, the carrier consists furthermore of at least 40 wt.-%, more preferably of at least 50 wt.-%, most preferably of at least 55 wt.-% of water, such as in particular of about 55 to about 90 wt.-% of water.

The topical compositions according to the present invention are preferably prepared by admixing the saccharide isomerate with all the definitions and preferences as given herein with/into a cosmetically acceptable carrier.

The use of saccharide isomerate according to the present invention may be therapeutic or non-therapeutic. Preferably, the use is non-therapeutic, i.e. for cosmetic application. Thus, preferably in all embodiments the topical compositions are cosmetic (non-therapeutic) compositions.

The term “cosmetic composition” as used in the present application refers to cosmetic compositions as defined under the heading “Kosmetika” in Rompp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4th edition, 1992.

Suitable composition according to the invention are leave-on or rinse-off products, and include any product applied to the human body, preferably for cleansing skin. Thus, the use is preferably by way of incorporation in a rinse-off composition.

Rinse-off as used herein is defined as per the Regulation (EC) No. 1223/2009 of the European Parliament and of the Council of 30 Nov. 2009 on cosmetic products (recast), i.e. a cosmetic product or composition is a rinse-off one when it is intended to be removed after application on skin, hair or mucous membranes of a human subject.

The rinse-off cosmetic composition presently disclosed may be a rinse-off, cosmetic cleansing composition, for example a rinse-off, cosmetic, personal care, cleansing composition. Examples of the rinse-off cosmetic composition (hereinafter also “the composition”) are a shower gel composition, a liquid soap, a body wash and a shampoo. The composition is not edible or comestible, like toothpastes.

Thus, in a particular embodiment, the present invention relates to a method according to the present invention which method comprises the step of (i) applying the rinse-off composition to the body skin, preferably the torso, the arms and/or the legs, followed by (ii) rinsing off the composition with water.

The composition can be in the form of a liquid, lotion, cream, foam, scrub, gel, soap bar or toner, or applied with an implement or via a face mask, pad or patch. Non-limiting examples of such compositions include leave-on skin lotions and creams, shampoos, conditioners, shower gels, face wash's, body wash's, toilet bars, antiperspirants, deodorants, depilatories, lipsticks, foundations, mascara, sunless tanners and sunscreen lotions.

The compositions of the invention (including the carrier) may comprise conventional adjuvants and additives, such as preservatives/antioxidants, fatty substances/oils, organic solvents, silicones, thickeners, softeners, emulsifiers, antifoaming agents, soaps, detergents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may also comprise further cosmetically active ingredients conventionally used in cosmetic compositions. Exemplary active ingredients encompass UV-filters, agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the active ingredients as well as the excipients, diluents, adjuvants, additives etc. can, based on the desired product form and application, easily be determined by the skilled person. The additional ingredients can either be added to the oily phase, the aqueous phase or separately as deemed appropriate.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

If the composition is an emulsion, such as in particular an O/W-, W/O-, Si/W-, W/Si-, O/W/O-, W/O/W- or a pickering emulsion, then the amount of the oily phase present in such cosmetic emulsions is preferably at least 10 wt.-%, such as in the range of 10 to 60 wt.-%, preferably in the range of 15 to 50 wt.-%, most preferably in the range of 15 to 40 wt.-%, based on the total weight of the composition.

In one embodiment, the compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the composition according to the invention is an O/W emulsion, then it contains advantageously at least one O/W- or Si/W-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd.), diethanolamine cetyl phosphate (e.g. as Amphisol® DEA from DSM Nutritional Products Ltd.), potassium cetyl phosphate (e.g. as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are polyalkylene glycol ethers, sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt. %, in particular in the range of 0.5 to 6 wt.-%, such as more in particular in the range of 0.5 to 5 wt.-%, such as most in particular in the range of 1 to 4 wt.-%, based on the total weight of the composition.

Particular suitable O/W emulsifiers to be used in the compositions according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate as well as polyalkylene glycol ethers such as in particular polyethylene stearyl ethers such as Steareth-2 and Steareth-21.

A particular suitable class of 0/W emulsifier to be used in the compositions according to the invention are polyalkyleneglycolethers. Particularly preferred 0/W emulsifier in all embodiments of the present invention are the stearyl ethers of polyethyleneglycol, such as most preferably Steareth-2 (Polyoxyethylen (2) stearylether) or Steareth-21 (Polyoxyethylen (21) stearylether) as well as mixtures thereof. Such polyalkyleneglycolethers emulsifiers are e.g. commercially available under the Brij tradename at Croda.

The cosmetic compositions according to the present invention advantageously comprise a preservative. Particular suitable preservatives in all embodiments of the present invention are phenoxyethanol and ethylhexylglycerin as well as mixtures thereof. When present, the preservative is preferably used in an amount of 0.1 to 2 wt.-%, more preferably in an amount of 0.5 to 1.5 wt.-%, based on the total weight of the composition.

The compositions according to the invention in general have a pH in the range of 3 to 10, preferably a pH in the range of 4 to 8 and most preferably a pH in the range of 5 to 8. The pH can easily be adjusted as desired with suitable acids, such as e.g. citric acid, or bases, such as sodium hydroxide (e.g. as aqueous solution), triethanolamine (TEA Care), Tromethamine (Trizma Base) and Aminomethyl Propanol (AMP-Ultra PC 2000), according to standard methods in the art.

Preferred topical compositions according to the present invention are rinse-off preparations, which are intended/required to be removed from the body by washing with solvent, preferably water, after the application of said composition.

Preferred rinse-off compositions in all embodiments according to the present invention are detergent rinse-off compositions, which are used for cleansing the skin. Such detergent rinse-off composition can either be solid such as e.g. in the form of a soap bar or in a liquid form such as e.g. a shower gel, a wash gel, a hair shampoo, a body shampoo, a foam bath, a shower bath or a shaving preparation or in the form of a foam such as e.g. a shaving foam. It is well understood by a person skilled in the art, that in case of detergent rinse-off compositions, the cosmetically acceptable carrier further comprises at least one surfactant and/or at least one soap.

Particularly preferred detergent rinse-off compositions according to the present invention are liquid detergent rinse of compositions.

Preferably, in all embodiments of the present invention, the detergent rinse-off compositions according to the present invention consist essentially of a saccharide isomerate, water, at least one, preferably several soaps and/or surfactants, moisturizers, chelating agents, basifying or acidifying agents, actives, solubilizers, pearlescent or opacifying agents, thickening agents, humectants, and additives which enhance their appearance, feel and fragrance, such as colorants, fragrances, preservatives, and the like.

The water content of the detergent rinse-off compositions according to the present invention is preferably selected in the range from 40 to 70 wt.-%, more preferably in the range from 45 to 65 wt.-%, most preferably in the range from 50 to 60 wt.-%.

The term “soap” is used herein in its popular sense, i.e., it refers to the alkali metal or alkanol ammonium salts of aliphatic, alkane- or alkene monocarboxylic acids as well as mixtures thereof. Sodium, potassium, magnesium, mono-, di- and tri-ethanol ammonium cations, or combinations thereof, are particularly suitable for the purposes of this invention, preferably, however, sodium or potassium soaps are used.

Particularly preferred soaps for the purpose of the present invention are the well-known alkali metal salts such as in particular the sodium and/or potassium salts of natural or synthetic aliphatic (alkanoic or alkenoic) acids having from about 8 to 22 carbon atoms, preferably from about 8 to about 20 carbon atoms, most preferably from about 10 to about 18 carbon atoms. Even more preferred are the salts of the respective saturated (alkanoic acid) acids.

It is furthermore preferred, that the soaps used in the detergent composition according to the present invention comprise at least 85% of fatty acids having from 12 to 18 carbon atoms.

Particularly preferred soaps to be used in the detergent compositions of the present invention are the sodium and/or potassium salts of stearic acid, lauric acid, myristic acid, oleic acid and palmitic acid.

It is well understood that the soap can be used as such, or can be formed ‘in situ’ in the detergent rinse-off composition by adding the respective acid and the respective base to the composition.

The amount of the at least one soap in the detergent rinse-off compositions according to the present invention may easily be selected by a person skilled in the art, such as in the range from 3 to 95 wt.-%. It is well understood by a person skilled in the art, that the amount is strongly dependent on the type of detergent rinse-off composition, i.e. if the detergent rinse-off composition is a soap bar the concentration is selected in the range of 50-95 wt.-%, while if the composition is a liquid (aqueous) rinse-off composition the concentration is selected in the range of 15 to 50 wt.-%, preferably in the range from 20 to 40 wt.-%, most preferably in the range from 25 to 35 wt.-%, based on the total weight of the detergent rinse-off composition.

Particularly preferred soaps to be used in detergent the rinse-off compositions according to the present invention are the sodium and/or potassium salts of lauric acid, stearic acid and myristic acid as well as mixtures thereof.

Particularly suitable surfactants to be used in the detergent rinse-off compositions according to the present invention, such as in particular in the liquid detergent rinse-off compositions are anionic, cationic, non-ionic and/or amphoteric surfactants to form a surfactant mixture.

Suitable anionic surfactants to be included into the detergent rinse-off compositions according to the invention include, but are not limited to aliphatic sulphate, aliphatic sulfonate (e.g., C₈ to C₂₂ sulfonate or disulfonate), aromatic sulfonate (e.g., alkyl benzene sulfonate), alkyl sulfosuccinates, alkyl and acyl taurates, alkyl and acyl sarcosinates, sulfoacetates, alkyl phosphates, carboxylate and isethionates as well as mixtures thereof.

Particularly suitable anionic surfactants to be used for the purpose of the present invention are alkyl sulfates such as preferably sodium, triethanolamine or ammonium lauryl sulfates; alkyl ether sulfates (or Alkyl PEG-n sulfates) such as preferably sodium or ammonium lauryl ether sulfate, laureth sulfate, sodium C₂₋₁₅ pareth sulfate; alkyl amido ether sulfates; alkylaryl polyether sulfates; monoglycerides sulfates; acyl isethionate salts such as preferably sodium acylisethionate, sodium cocoyl isethionate; alkylaryl sulfonates salts such as preferably sodium alkylbenzene sulfonate and/or sodium dodecylbenzene sulfonate; alkyl sulfonates salts such as preferably sodium alkenyl sulfonate (sodium C₁₂₋₁₄ olefin sulfonate), sodium alkylglyceride sulfonate (sodium cocomonoglyceride olefin sulfonate), sodium alkylether sulfonate (sodium C₁₂₋₁₅ pareth-15 sulfonate) and/or sodium lauryl sulfoacetate; (di)sodium sulfosuccinates such as preferably sodium dialkyl sulfosuccinate (dioctyl sodium sulfosuccinate), disodium alkyl PEG-n sulfosuccinate, disodium alkylamido PEG-n sulfosuccinate (disodium oleamido MEA-sulfosuccinate), disodium alkylsulfosuccinate; alkyl phosphates (mono-esters) such as preferably TEA monolauryl phosphate; PEG-n alkyl phosphates such as preferably DEA oleth-10 phosphate; di PEG-n alkyl phosphates (di-esters) such as preferably dilaureth-4 phosphate; phospholipids (tri-esters) such as preferably lecithin; carboxylic acids ester, such as preferably mono-ester of di- or tri-carboxylic acids such as lactylates (sodium acyllactylate, calcium stearoyl lactylate), laureth-6 citrate, dinonoxynol-9 citrate; ether carboxylic acids such as preferably sodium PEG-n alkyl carboxylates, sodium trideceth-13 carbon/late, nonoynol-8 carboxylic acid, alkyl C₆-C₂₄ ether carboxylates polyoxyalkylenated; acyl glutamates such as preferably di-TEA palmitoyl aspartate and sodium hydrogenated tallow glutamate; Acyl peptides with various amino acids side groups such as preferably palmitoyl hydrolysed milk protein, sodium cocoyl hydrolysed soy protein, TEA-cocoyl hydrolysed collagen or other acyl hydrolysed protein salts; sarcosinates or acyl sarcosides such as preferably myristoyl sarcosine, TEA-lauroyl sarcosinate; as well as taurates and sodium methyl acyltaurates such as preferably sodium lauroyl taurate, sodium methyl cocoyl taurate.

Particularly suitable non-ionic surfactants to be used for the purpose of the present invention encompass ethers comprising aliphatic (C₆-C₁₈) primary or secondary linear or branched chain acids, alcohols or phenols which possess no functional grouping other than the terminal OH group of the Polyoxyethylenated (POE) chain as well as ethoxylated alcohols and propoxylated POE ethers such as preferably PEG ethers, PPG ethers, propylene glycol alkyl POE-n ethers; alkyl polyglucosides of the general formula CₙH₂ₙ₊₁O(C₆H₁₀O₅)ₓH, with x being 1 to 4 such as preferably decyl glucoside, and lauryl glucoside; alkanolamides such as preferably N-acyl derivatives of monoethanolamine (MEA) and diethanolamine (DEA), ethoxylated or not; such as preferably PEG-n acylamides, coco mono- or di-ethanolamide, palmamide MEA, Acylamide DEA; esters such as preferably ethoxylated fatty acids; mono- and di-esters of fatty acids with ethylene oxide or polyethylene glycol, PEG-n acylate and diacylate such as PEG-8 laurate, PEG-8 dilaurate, PEG-100 stearate, PEG-150 distearate, ethoxylated glycerides such as preferably PEG-n glyceryl acylate, PEG-4 castor oil, PEG-120 glyceryl stearate, triolein PEG-6 esters, glycol esters and derivatives, mono-esters of either ethylene or propylene glycol such as preferably glycol acylate or propylene glycol acylate, monoglycerides such as glyceryl myristate or stearate, glyceryl palmitate lactate, polyglyceryl esters such as polyglyceryl-n acylate or polyglyceryl-n alkyl ether, sorbitan/sorbitol esters such as preferably acetylated sorbitan ethoxylated or not, polysorbate-n, sorbitan sequiisostearate, alkyl carbohydrates esters or sucrose esters resulting from trans-esterification of sucrose with fatty acid methyl esters or triglycerides such as preferably alkylpolysaccharides; as well as amine oxides such as preferably cocoamidopropyl amine oxide and lauramine oxide. A particularly preferred group of non-ionic surfactant to be used in the rinse-off compositions according to the present invention are the alkyl polyglucosides such as lauryl glucoside, PEG-n acylate and diacylates such as PEG 100 stearate and glyceryl stearate.

Particularly suitable zwitterionic and amphoteric surfactants according to the present invention encompass secondary or tertiary aliphatic amine derivatives with an aliphatic chain, linear or branched, containing at least 8 to 22 carbon atoms and one anionic group selected from the group of carbon/late, sulfonate, sulfate, phosphate or phosphonate; acyl/dialkyl ethylenediamines such as preferably acylamphoacetate, disodium acylamphodipropionate, sodium acylamphohydroxypropylsulfonate, disodium acylamphodiacetate, sodium acylamphopropionate and wherein the acyl group represents either an alkyl or alkenyl group which can be mon- or polyunsaturated and contains from 5 to 29 carbon atoms; N-alkyl amino acids or imino diacids such as preferably aminopropyl alkylglutamide, alkylaminopropionic acid, sodium alkylimino propionate, alkyl glycinates and carboxyglycinates, sodium cocoglycinates; sas well as betaines such as preferably alkyl (C₈-C₂₀) betaines, alkyl amidopropyl betaines (cocamidopropyl betaines), alkyl (C₈-C₂₀) amidoalkyl (C₁-C₆) betaines, alkyl sulphobetaines and alkyl (C₈-C₂₀) amidoalkyl (C₁-C₈) sulphobetaines.

Particularly suitable cationic surfactants according to the present invention encompass alkylamines such as preferably dimethyl alkylamine (dimethyl lauramine), dihydroxyethyl alkylamine dioleate, acylamidopropyldimethylamine lactate (cocamidopropyl dimethylamine lactate); alkyl imidazolines such as preferably alkyl hydroxyethyl imidazoline, Ethylhydroxymethyl oleyl oxazoline, alkyl aminoethyl imidazoline; ethoxylated alkylamines such as preferably PEG-n alkylamines, PEG-n Alkylaminopropylamine, poloxamine; quaternary compounds such as preferably tetraalkylammonium salts; alkyl trimonium chloride, PEG-n alkylmonium chloride, dialkyldimonium chloride (hydroxyethyl cetyldimonium chloride), alkylamidopropyl alkyldimonium tosylate (Cocamidopropyl ethyldimonium ethosulfate), PEG-n Acylmethyldiethonium methosulfate, dialkyl hydroxypropylmonium methosulfate, and alkyldimonium hydroxypropyl protein hydrolysate (Cocodimonium hydroxypropyl hydrolysed hair keratin).

Particularly preferred surfactants to be used in the rinse-off compositions according to the present invention are selected from the group glucosides such as e.g. lauroyl glucosides, arachidyl glucoside, caprylyl/capryl glucoside and coco-glucoside, PEG-n acylate and diacylate such as e.g. PEG-8 laurate, PEG-8 dilaurate, PEG-100 Stearate, monoglycerides such as glyceryl myristate or stearate as well as mixtures thereof.

The amount of the at least surfactant in the detergent rinse-off compositions according to the present invention is preferably selected in the range of 1 to 30 wt.-%, preferably 2.5 to 10 wt.-%, most preferably 5 to 15 wt.-%, based on the total weight of the detergent rinse-off composition. Preferably, however, the amount of the surfactant in the rinse-off composition is selected in the range from 15 to 50 wt.-%, preferably in the range from 20 to 40 wt.-%, most preferably in the range from 25 to 35 wt.-%, based on the total weight of the detergent rinse-off composition.

In a particular preferred embodiment, the detergent rinse-off composition according to the present invention comprises a mixture of at least one, preferably several soaps and at least one surfactant with all the definitions and preferences as given herein. Even more preferably the total amount of the mixture of soap(s) and surfactant(s) is selected in the range of 15 to 50 wt.-%, preferably in the range from 20 to 40 wt.-%, most preferably in the range from 25 to 35 wt.-%, based on the total weight of the detergent rinse-off composition. It is then even more preferred that the soap constitutes greater than 60 wt.-%, more preferably greater than 65 wt.-%, most preferably greater than 70 wt.-% of said mixture.

Suitable chelating agents to be incorporated into the detergent rinse-off compositions according to the present invention encompass those which are capable of protecting and preserving the compositions of this invention. Preferably, the chelating agent is ethylenediamine tetraacetic acid (“EDTA”), and more preferably is tetrasodium EDTA, available commercially from Dow Chemical Company of Midland, Michigan under the tradename, “Versene 100XL” or even disodium EDTA, commercially available from BASF under tradename “EDETA BD”.

Others suitable chelating agents include Phytic acid and its sodium salts, gluconic acid and its sodium salts and etidronic acid and its sodium salts, phosphoric acid and its sodium salts, gluconic acid and its sodium salts, oxalic acid, citric acid, lauryl alcohol diphosphonic acid, tetrasodium glutamate diacetate, trisodium dicarboxymethyl alaninate, trisodium ethylenediamine disuccinate.

The amount of the chelating agent is preferably selected in the range from 0.01 to 1 wt.-%, preferably in the range from 0.1 to 0.75 wt.-%, most preferably in the range from 0.25 to 0.75 wt.-%, based on the total weight of the detergent rinse-off composition.

The acidifying agents can be, for example, mineral or organic acids, for instance hydrochloric acid, orthophosphoric acid, carboxylic acids, for instance tartaric acid, citric acid, lactic acid, or sulphonic acids.

Among the basifying agents which may be mentioned, for example, are the alkaline or earth alkaline salts such as sodium or potassium hydroxide, alkali metal carbonates, as well as alkanolamines such as monoethanolamine, diethanolamine and triethanolamine

The basifying or acidifying agent is used to adjust the pH of the rinse-off compositions. In a preferred embodiment, the pH of the detergent rinse-off composition according to the present invention is adjusted with the basifying or acidifying agent to be in the range from about 4.5 to about 10.5, and more preferably from about 5.0 to about 10.0.

Furthermore, the amount of the basifying or acidifying agent in the detergent rinse-off composition according to the present invention is preferably at least 0.0001 wt.-%, such as e.g. in the range from 0.01 to 6 wt.-%, in particular in the range from 3 to 5 wt.-%.

The detergent compositions of the present invention may also include one or more optional ingredients such as a pearlescent or opacifying agent, a thickening agent, humectants, and additives which enhance their appearance, feel and fragrance, such as colorants, fragrances, preservatives, and the like.

Commercially available pearlescent or opacifying agents which are capable of suspending water insoluble additives and/or which tend to indicate to consumers that the resultant product is a detergent composition are suitable for use in this invention. The pearlescent or opacifying agent may be present in an amount, based upon the total weight of the composition, of from about 1 wt.-% to about 10 wt.-%, preferably from about 1.5 wt.-% to about 7 wt.-%, and more preferably, from about 2 wt.-% to about 5 wt.-%.

Examples of suitable pearlescent or opacifying agents include, but are not limited to mono or diesters of (a) fatty acids having from about 16 to about 22 carbon atoms and (b) either ethylene or propylene glycol mono or diesters of (a) fatty acids having from about 16 to about 22 carbon atoms, (b) a polyalkylene glycol of the formula: HO-(JO)a-H, wherein J is an alkylene group having from about 2 to about 3 carbon atoms and a is 2 or 3; fatty alcohols containing from about 16 to about 22 carbon atoms; fatty esters of the formula: KCOOCH2L, wherein K and L independently contain from about 15 to about 21 carbon atoms; inorganic solids insoluble in the detergent composition, and mixtures thereof.

The pearlescent or opacifying agent may be introduced to the detergent composition as a pre-formed, stabilized aqueous dispersion, such as that commercially available from Henkel Corporation of Hoboken, New Jersey under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and preferably is in a weight percent ratio of from about 25 to about 30:about 3 to about 15:about 20 to about 25, respectively.

Commercially available thickening agents, which are capable of imparting the appropriate viscosity to the detergent rinse-off compositions are suitable for use in this invention. If used, the thickener should be present in the compositions in an amount sufficient to raise the Brookfield viscosity of the composition to a value of between about 500 to about 10,000 centipoise. Examples of suitable thickening agents nonexclusively include: mono or diesters of 1) polyethylene glycol of formula: HO—(CH₂CH₂O)_{z}H, wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerin; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Illinois or from Comiel, S.p.A. of Bologna, Italy under the tradename, “PEG 6000 DS”.

The amount of thickener(s) in the detergent rinse-off composition is preferably selected in the range from 0 to 7 wt.-%, preferably from 1 to 5 wt.-%, most preferably from 2 to 4 wt.-%, based on the total weight of the detergent rinse-off composition.

Commercially available humectants, which are capable of providing moisturization and conditioning properties to the detergent composition, are suitable for use in the present invention. Examples of suitable humectants nonexclusively include: 1) water soluble liquid polyols selected from the group comprising glycerol, propylene glycol, 1,3-propanediol, hexylene glycol, butylene glycol, dipropylene glycol, and mixtures thereof; 2) polyalkylene glycol of the formula: HO—(R″O)b-H, wherein R″ is an alkylene group having from about 2 to about 3 carbon atoms and b is an integer of from about 2 to about 10; 3) polyethylene glycol ether of methyl glucose of formula CH₃-C₆H₁₀O₅—(OCH₂CH₂)c-OH, wherein c is an integer from about 5 to about 25; 4) urea; and 5) mixtures thereof, with glycerol or PEG-32 being the preferred humectant.

Preferably, in all embodiments, the detergent rinse-off composition comprise at least one humectant. The amount of the at least on humectant, if present in the detergent composition is preferably selected in the range from 0 to 90 wt.-%, preferably from 5 to 40 wt.-%, most preferably from 15 to 30 wt.-%, based on the total weight of the detergent rinse-off composition. Further suitable ranges encompass ranges from 1 to 10 wt.-%, from 2 to 8 wt.-% and from 2.5 to 5 wt.-%, based on the total weight of the detergent rinse-off composition.

Suitable preservatives to be included into the detergent rinse-off compositions according to the present invention include Quaternium-15, available commercially as “Dowicil 200” from the Dow Chemical Corporation of Midland, Michigan, and are present in the composition in an amount ranging from about 0 to 5.0 wt.-%, preferably from about 0.05 to 2 wt.-%, most preferably from about 0.1 to 1.5 wt.-%, based on the total weight of the detergent composition.

The detergent composition of the present invention may be used on the body in conjunction with any personal cleansing implement known in the art such as a washcloth, a mesh or apertured film, pouf, sponge, brush and the like. The composition may be marketed together with one or more of such implements in a kit.

The compositions of the present invention may furthermore be “substantially free” of oils or silicones. As used herein, “substantially free” shall mean that the detergent rinse-off composition contains less than about 1 wt.-%, for example, less than about 0.5 wt.-% or less than about 0.2 wt.-% oils and/or silicones, based on the total weight of the detergent composition.

In another preferred embodiment, the detergent rinse-off compositions according to the present invention are (solid) soap bars.

Most preferred detergent rinse-off compositions according to the present invention consist essentially of a saccharide isomerate, water, at least one, preferably several soaps and/or surfactants and optionally moisturizer(s), chelating agent(s) and a basifying or acidifying agent with all the definitions and preferences as given herein.

The following examples are provided to further illustrate the compositions and effects of the present invention. These examples are illustrative only and are not intended to limit the scope of the invention in any way.

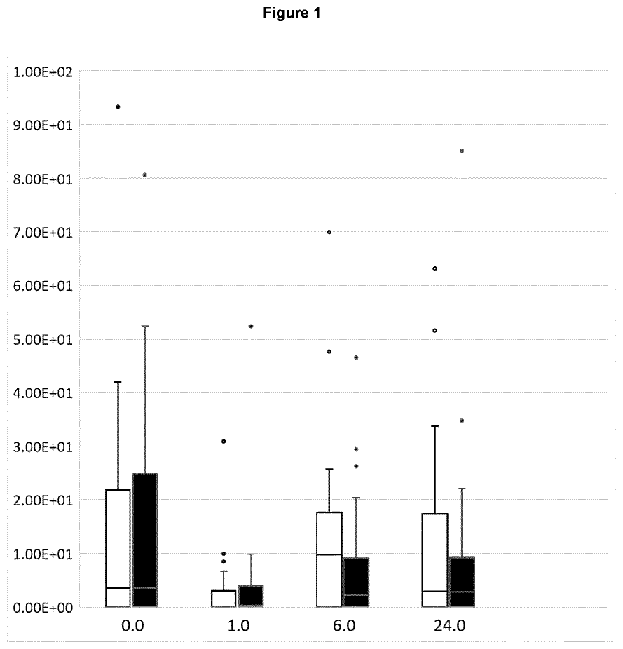

FIG. 1: qPCR assessment of the microbial load of S. epidermidis (time 0: black and white bar: before treatment on symmetrical positions of the arm volar forearms as outlined in the example; time 1.0 to 24.0 hours: white bars: placebo, black bars: treatment; y axis: quantity per μl of DNA, x axis: time (hours). For further details refer to the experimental part.

### EXAMPLE

### Example: In Vivo Study to Demonstrate Enrichment of P. marcusii on Skin Upon Use of Saccharide Isomerate on Body Skin

In a 7 days in vivo placebo-controlled clinical study (30 volunteers), a rinse-off body wash formulation containing 0.5% saccharide isomerate as outlined in table 1 was applied on volar forearms once during the first 24 hours and twice daily for the remaining days up to day 7. Microbiome samples were collected at different timepoints (T0, 1 h, 6 h, 24 h and at day 7) and 16S rRNA sequencing as well as qPCR were performed on the extracted DNA. The results obtained from the 16S rRNA sequencing where processed following the “reference frames” approach published by Morton et al. (Nat. Commun., 2019. 10(1): p. 2719). The method allows to assess microbiota changes and how these changes are associated with time and with the treatment. A software called Songbird is adopted to calculate differentials starting from relative abundance data coming from 16S sequencing. The differential values referred to time and treatment are reported in the table 2 below.

TABLE 1
Formulations

| | Base | |
|---|---|---|
| | formulation | Active |
| | (Placebo) | formulation |
| INCI Name | Wt.-% | Wt.-% |
| Aqua | Ad 100 | Ad 100 |
| Disodium EDTA, aqua | 0.05 | 0.05 |
| Lauryl glucoside (50-53% in water) | 4.00 | 4.00 |
| PEG-32 | 5.00 | 5.00 |
| Lauric acid | 3.30 | 3.30 |
| Stearic acid | 12.00 | 12.00 |
| Myristic acid | 11.50 | 11.50 |
| Glyceryl stearate, PEG-100 stearate (50:50) | 2.00 | 2.00 |
| Potassium hydroxide | 4.65 | 4.65 |
| Saccharide isomerate, aqua, citric acid, | 0.00 | 0.50 |
| sodium citrate | | |

TABLE 2
The “reference frames” analysis led to the following results

| Time points | | | | |
|---|---|---|---|---|
| comparison | Intercept | Treatment | Time | Species |
| T0-1 h | 2.841325603 | 0.455594273 | −21.2721267 | P. marcusii |
| T0-1 h | 2.006193614 | −0.441441028 | −3.639258889 | R. mucilaginosa |
| T0-T1 h | 1.495349384 | −0.086419974 | 11.30036876 | S. epidermidis |
| T0-1 h-6 h | 2.653772826 | 0.368095544 | −2.9598190978 | P. marcusii |
| T0-1 h-6 h | 1.220426777 | −0.66170466 | −0.351324101 | R. mucilaginosa |
| T0-1 h-6 h | 2.964037099 | −0.207998651 | −0.183416148 | S. epidermidis |
| T0-D 7 | 0.133625727 | 0.695163753 | −0.075945504 | P. marcusii |
| T0-D 7 | 1.72537617 | −0.5798218 | 0.015160795 | R. mucilaginosa |
| T0-D 7 | −0.02278974 | −0.85521415 | 0.338541217 | B. casei |
| T0-D 7 | 1.920367282 | 0.035157144 | 0.014309752 | S. epidermidis |
| 24 h-D 7 | −2.65664782 | 0.624407053 | −0.039027537 | P. marcusii |
| 24 h-D 7 | −0.5959424 | −0.81193221 | 0.374109504 | B. casei |
| 24 h-D 7 | 1.62424944 | 0.006250679 | 0.014235535 | S. epidermidis |

The column “Time points comparison” indicates the timeframe in which the microbial changes are evaluated. Example: T0-1 h-6 h indicates that the microbial shifts are evaluated in the time window going from the beginning of the study (TO) up to the 6 hours timepoint after the first product application. The column “Intercept” indicates the starting point for each microorganism. A positive value means that the microorganism of interest was present to a certain extent on the skin before the evaluation. The column “Treatment” refers to if and to what extent a specific microorganism is associated with the active treatment. The values reported are the result of the differential made by the software by using placebo-valued samples as a reference. Therefore, a positive value indicates positive association with the active treatment. The “Time” column, suggests how specific microorganisms are associated with time, independently of the treatment. A positive value means that the microbe tend to increase its abundance over time.

Overall, the results show a strong association of Paracoccus marcusii with the active treatment containing saccharide isomerate, at all the timepoints considered. In contrast, so called “coryneforms” such as Rothia mucilaginosa and Brevibacterium casei showed to be negatively associated with the active treatment at the timepoints considered. The positive effect of saccharide isomerate on the Paracoccus marcusii enrichment is further strengthened by the fact that this bacterium would normally decrease its abundance on skin over time as shown in the table, likely because of the skin cleansing procedure. Surprisingly, S. epidermidis, a common skin commensal, was negatively associated with the active treatment containing saccharide isomerate especially during the first 6 hours and its differential abundance was not particularly supported by the treatment in the following time points assessed (slight positive values indicating anyway a very weak association at 24 h and D7).

To further strengthen our conclusions, this result was confirmed by qPCR assessment of the microbial load of S. epidermidis over 24 hours, as outlined in FIG. 1.

The data shows that the microbial load of S. epidermidis was reduced compared to baseline (T0), most likely because of the cleansing procedure as this reduction was observed in both placebo and active treatment groups. In the following time points there was no evidence of any growth boost compared to placebo by the treatment. These results align very well with the differential abundance analysis in which S. epidermidis was seen to be negatively associated with the active.

Taken together the data suggest that the rinse-off formulation containing saccharide isomerate selectively supports and enriches P. marcusii on the skin, without supporting other commensals like S. epidermidis, and furthermore interferes with the colonization of pathogenic coryneforms such as in particular R. mucilaginosa and B. casei.

### Comparative Example: In Vivo Study Demonstrating a Negative Association of P. marcusii Upon Use of Saccharide Isomerate on Face Skin

In a 28 days in vivo placebo-controlled clinical study, 6 Caucasian female volunteers with dry skin conditions, aged between 20 and 50 years old, applied a leave on hydrogel formulation containing 3% saccharide isomerate as outlined in table 1, which was applied twice daily on the face for 28 days. Microbiome samples were collected on the cheeks and on the forehead at different timepoints (TO, day 7 and day 28) and 16S rRNA sequencing was performed. The results obtained from the 16S rRNA sequencing where processed following the “reference frames” approach published by Morton et al. (Nat. Commun., 2019. 10(1): p. 2719). The method allows to assess microbiota changes and how these changes are associated with time and with the treatment. A software called Songbird is adopted to calculate differentials starting from relative abundance data coming from 16S sequencing. The differential values referred to time and treatment are reported in the table 2 below.

TABLE 2
Formulations

| | Base | |
|---|---|---|
| | formulation | Active |
| | (Placebo) | formulation |
| INCI Name | Wt.-% | Wt.-% |
| ACRYLATES/C10-30 ALKYL | 0.30 | 0.30 |
| ACRYLATE CROSSPOLYMER | | |
| AQUA | 98.54 | 95.54 |
| PHENOXYETHANOL, | 1.00 | 1.00 |
| ETHYLHEXYLGLYCERIN | | |
| SACCHARIDE ISOMERATE, AQUA, | 0.00 | 3.00 |
| CITRIC ACID, SODIUM CITRATE | | |
| AQUA, SODIUM HYDROXIDE | 0.16 | 0.16 |

TABLE 2
The “reference frames” analysis led to the following results

| Time points | | | | |
|---|---|---|---|---|
| comparison | Intercept | Treatment | Time | Species |
| T0-7 D | −0.359157702 | −2.488678243 | 0.205731614 | P. marcusii |
| T0-D 28 | 1.340011788 | −1.187325642 | −0.031220022 | P. marcusii |
| D 7-D 28 | 1.340011788 | −1.187325642 | −0.031220022 | P. marcusii |

The column “Time points comparison” indicates the timeframe in which the microbial changes are evaluated. Example: TO-7D indicates that the microbial shifts are evaluated in the time window going from the beginning of the study (TO) up to 7 days of product application. The column “Intercept” indicates the starting point for each microorganism. A positive value means that the microorganism of interest was present to a certain extent on the skin before the evaluation. The column “Treatment” refers to if and to what extent a specific microorganism is associated with the active treatment. The values reported are the result of the differential made by the software by using placebo-valued samples as a reference. Therefore, a positive value indicates positive association with the active treatment. The “Time” column, suggests how specific microorganisms are associated with time, independently of the treatment. A positive value means that the microbe tend to increase its abundance over time.

Surprisingly, in contrast to the data obtained on body, the results obtained on face show a strong negative association of Paracoccus marcusii with the active treatment containing saccharide isomerate, at all the timepoints considered.

1. A method to increase the differential abundance of P. marcusii in the skin microbiome of an individual, said method comprising the step of topically administering an effective amount of saccharide isomerate to an external surface of the body in need thereof and wherein the external surface of the human body is the body skin.

2. The method according to claim 1, to foster the supply of carotenoids to the external surface of the human body and/or the degradation of polyaromatic hydrocarbons after adsorption thereof onto the external surface of the human body

3. The method according to claim 2, wherein the carotenoid is astaxanthin.

4. The method according to claim 2, wherein the adsorption of the polyaromatic hydrocarbons is caused by environmental pollution.

5. The method according to claim 1, wherein the saccharide isomerate is administered in the form of a cosmetic composition comprising an effective amount of saccharide isomerate and a cosmetically acceptable carrier.

6. The method according to claim 5, wherein the amount of saccharide isomerate is selected in the range from 0.01 to 10 wt.-%, preferably in the range from 0.1 to 7.5 wt.-%, most preferably in the range from 0.2 to 5 wt.-%, based on the total weight of the cosmetic composition.

7. The method according to claim 5, wherein the cosmetic composition is a rinse-off composition.

8. The method according to claim 7, wherein the rinse-off composition is a detergent rinse-off composition further comprising water and at least one surfactant and/or at least one soap.

9. The method according to claim 8, wherein the detergent rinse-off composition consists essentially of the saccharide isomerate, water, at least one, preferably several soaps and/or surfactants, at least one moisturizer, at least one chelating agent and at least one basifying or acidifying agent.

10. The method according to claim 1, comprising the step of (i) applying the rinse-off composition to the body skin preferably the torso, the arms and/or the legs, followed by (ii) rinsing it off with water.

11. The method according to claim 1, wherein at the same time the differential abundance of coryneforms, preferably of B. casei and/or R. Mucilaginosa in the skin microbiome is reduced.

12. A method for providing protection to an external surface of the human body which is the body skin by increasing the differential abundance of P. marcusii thereon, said method comprising the step of applying saccharide isomerate to said external surface.

13. A method to supply carotenoids, preferably astaxanthin to an external surface of the human body comprising body skin, said method encompassing the step of topically administering saccharide isomerate to said external surface in need thereof.

14. Use of saccharide isomerate for increasing the differential abundance of P. marcusii in the skin microbiome of the body skin of an individual in need thereof.

15. The use according to claim 13, wherein concomitantly the differential abundance of coryneforms, preferably of B. casei and/or R. mucilaginosa is reduced.
